SEQUENCE LISTING (1 sequence)

SEQ ID NO: 1 - protein, 9 aa, homo sapiens
  misc_feature (2)..(2): Xaa can be any naturally occurring amino acid
  misc_feature (8)..(8): Xaa can be any naturally occurring amino acid
DXNDNAPXF

(19) United States
(12) Patent Application Publication
(10) Pub. No.: US 2019/0040393 A1
(43) Pub. Date: Feb. 7, 2019

(54) Managing Intercellular Communications

(71) Applicant: Richard Postrel, Miami Beach, FL (US)

(72) Inventor: Richard Postrel, Miami Beach, FL (US)

(21) Appl. No.: 15/952,218

(22) Filed: Apr. 12, 2018

Related U.S. Application Data
(60) Provisional application No. 62/540,833, filed on Aug. 3, 2017.

(51) Int. Cl. C12N 15/113; A61K 9/107; A61K 31/428; A61K 38/10; A61K 31/18; A61K 31/4184; A61K 31/436; A61K 45/06; A61K 31/7088; A61K 31/16

(52) U.S. Cl. CPC C12N 15/1132; C12N 2320/35; A61K 31/428; A61K 38/10; A61K 31/18; A61K 31/4184; A61K 31/436; A61K 45/06; A61K 31/7088; C12N 15/1133; C12N 15/1131; A61K 31/16; C12N 15/113; C12N 15/1137; C12N 2320/32; A61K 9/1075

(57) ABSTRACT

The present invention describes natural, artificial, and/or enhanced methods for controlling intercellular and intracellular communications involving complex biological, biochemical reactions and the like. It transforms intercellular data into actionable and remedial intervention when reactions or existing intercellular interactions are not in synchrony with the normal or historic demands of the macroorganism. The invention controls intercellular communication systems by initiating, enhancing, inhibiting or preventing material and information transfer through tunneling nanotubules (TNTs) and/or through exosomal exchanges. Although TNTs are generally beneficial, the present invention especially recognizes that several diseases have developed patterns that take advantage of or make use of TNTs to propagate disease by direct intercellular connection between neighbor cells. Inhibiting TNT formation can prevent disease spread while other defenses such as immune system forming protective antibodies comes to fruition. An alternative or enhanced practice of the invention manages exosome production to deliver deleterious substance to prevent infected or otherwise unnecessary cells from commandeering resources for their benefit to the macroorganism's detriment. The exosomes may be used independently but preferably together with the TNT therapy. The exosomes can be engineered to specifically target a receptor protein or specific cell for delivery of the exosomal package which may include small molecule, cofactor ion, mRNA, miRNA, protein, pharmaceutical and/or other desired composition that can be incorporated in the exosomal membrane or its inner content. Optional assessment tools included bioassays and electronic monitoring devices. Proper management of TNT and/or exosome intercellular communication results in optimization of the macroorganism's metabolism and quality of life and/or minimizing metabolism compromising and/or disease sustaining attributes.

The present invention describes natural, artificial, and/or enhanced methods for controlling intercellular and intracellular communications involving complex biological, biochemical reactions and the like. The invention transforms intercellular data into actionable and remedial intervention when reactions or existing intercellular interactions are not in synchrony with the normal or historic demands of the macroorganism.

Humans like all other animals and plants are made of the same thing, cells. The adult human body has about 3-4 trillion cells. We have over 200 general types of cells, each kind of doing their specific thing. Each cell comprises genes for over 2500 enzymes, with many of these enzymes capable of multiple related reactions and many of these enzymes participating in a plurality of pathways. If each really did their own thing we would have chaos. The cells of humans and of all macroorganisms must cooperate so that they support the survival of the macroorganism. Cooperation requires a sense of what the other cells are doing so that each cell has its requirements met and its wastes removed. These processes require thousands of different enzymes to control biochemical reactions that sustain life.

At a first level of control a potential reaction in an off state can be turned on, for example through action of a receptor to synthesize or activate a transcription factor to start production of a new enzyme. This enzyme will then have cascading effects as its activities remove product and produce reactants that will affect other enzyme systems. The effects on other systems and pathways will not generally be an on-off switch, but will act as potentiometer up-regulating some systems, perhaps by making more substrate available, while own regulating others, perhaps though removing a common substrate or by creating a negative feedback. On a more complex scale the potentiation may include other enzymes such as kinase that modify proteins and their activities by attaching phosphate groups. With each reaction the system becomes more complex in a geometric or exponential fashion. All these reactions, potential or active, must be controlled in a balancing act involving thousands of chemical interactions necessary or advantageous for cell survival.

The cells of all macroorganisms and therefore are very small. Remember our bodies comprise several trillions of them. Most individual cells require a microscope to see their shapes and boundaries. And each of these cells derives from a division of an already existing cell. But in complex organisms like humans who each trace back to a single cell, the eventual progeny of that single cell will acquire vastly different appearances and functions.

While all the reactions within a cell had to be balanced to allow the cell to survive, the functions of the cell types and cells must also be balanced and the balancing between cells has to feedback to each cell's internal activities to support the growth and life of the macroorganism.

All parts of our bodies are made up of the cells performing their delegated function(s). As such no cell can be characterized as typical. Humans have about 200 different broad cell type classifications. And each of these cell types covering the variety of functions will share about twenty or so different intracellular structures or organelles. Yet despite these gross similarities, the different delegated functions require a distinct balancing act for each cell type to balance contributions, perhaps complete absence or disuse of one or more internal structure, but an extremely complex cooperative interaction with respect to those active in that cell type. Then each cell must balance its own reactions to meet the cell's needs and requirements of the organism.

The intracellular balancing act is complex enough, but when compounded by intercellular needs to address life requirements of the macroorganism, the coordination is much more difficult. Functions and reactions within the cell are constrained by the cell's plasma membrane which isolates the cell's interior from the surroundings. The plasma membrane acts as a gatekeeper, only allowing certain molecules generally defined by membrane bound receptors, to enter or exit the cell. The organelles, enzymes, transport proteins, and cytoskeletal components have direct access to each other. Balancing the functions and reactions between cells however, requires a communication between cells.

Three levels of intercellular communication through direct contact are possible. Cells directly abutting each other can “talk” across their membranes, a first cell signaling a second cell how it should proceed by affecting a plasma membrane messenger protein on the second cell allowing the second cell to react in similar fashion as a cell would react to a circulating extracellular signal. A second form of direct messaging has the first cell transmitting the message through the juxtaposed membranes with the actual ions or small molecules switching from one cell to the other. A third type of direct contact intercellular communication actually can occur between cells of some distance from one another. Cells can reach out to near neighbor cells with a pipe-like structure or tunnel between the cells. These tubular tunnels cam extend several cell diameters and can be of a size sufficient for transporting not only ions and small molecules, but substances as large as organelles, including, but not limited to: mitochondria, lysosomes, endoplasmic reticulum, Golgi, endosome and intracellular as well as extracellular amyloid β, etc.

At a first level, the intercellular communication can be between cells adjacent to or in direct physical contact with each other. Direct physical contact, intercellular adhesion, is maintained by membrane proteins binding—that is, attaching to—one another. Intercell adhesion is a vital occurrence for maintenance of the body's structure and integrity. Loss of adhesion would remove extracellular physical barriers and thus would have profound effects on cell shape and structure, the cell's functions and the cell's interactions/communications with other cells.

There are four major classes of adhesion proteins in mammals.

A. Selectins are involved in the initiation of inflammatory processes, for example, leukocyte collaborations with vascular endothelium. The three types of selectins identified in humans are: L-selectin, featured in lymphocytes, monocytes and neutrophils; P-selectin featured in platelets and endothelium; and E-selectin featured in endothelium. Each has an amino-terminal lectin extracellular domain bound to a carbohydrate ligand, a growth factor-like domain (EGF) and several short repeat units to effect complementary protein binding.

B. Integrins are adhesion receptors that transport signals across the plasma membrane. Many integrins exist with different preferred ligands and intracellular messaging. Each integrin comprises an α and a β subunit. There are 18 distinct a subunit genes and 8 distinct β subunit genes, many of which having splice variants. Integrin binding is under control of divalent ion cofactors such as Ca⁺⁺ and Mg⁺. Integrins are involved in pathways that mediate cellular signals involved in processes or functions including, but not limited to: cell cycle, organization of the intracellular cytoskeleton, and movement of new receptors to the cell membrane. These integrin molecules are thus integral to intercellular signaling interactions.

C. Cadherins are calcium-dependent adhesion molecules. Cadherins are extremely important in processes of morphogenesis as in fetal development. Together with an α-β catenin complex, cadherins bind to the microfilaments of the cytoskeleton. At least one cadherin is implicated in human asthma.

D. Immunoglobulin superfamily comprises a group of calcium independent proteins that bind between cells of the same or of different types. Same-type adhesion involves the immunoglobulin-like domains on the cell surface that bind to immunoglobulin-like domains on an opposing cell's surface. Different-type adhesion involves to binding of the immunoglobulin-like domains to integrins and/or carbohydrates.

These adhesion protein interactions involve interaction of one cell's membrane outer layer or transmembrane protein at the surface of the membrane outer layer of a second cell without any transfer or exchange of intracellular material between cells.

Cells in direct contact with one another have abilities for transfers of small molecules across the two membranes. One structure cells use is the gap junction. Gap junctions are pore structures that directly connect the cytoplasm of two cells. This allows passage of small molecules, ions and electrical impulses to directly pass from the cytoplasm of one to the other. As a rule, a gap junction is available for molecules up to about 485 Daltons. Some gap junctions may impart an electrical charge selectivity. The membrane remains impervious to most proteins, nucleic acids and virtually all organelles.

Gap junctions occur throughout body tissues including especially the heart muscle where they are instrumental in coordinated conductivity of the action potential throughout the heart muscle. Gap junctions are also present in other less active tissues including for example, skin. Gap-junction transmission is limited by the pore sizes of the proteins of the junction with small second messengers, e.g., inositol triphosphate (IP3) and Ca⁺⁺ representative of their activities. The transmission through gap junctions avoids losses into interstitial space.

Cell adhesion is one factor controlling cancer. Cancer can involve loss of cell-cell interaction that imposes growth controls on cells surrounded by like cells. In regular order contact inhibition resulting from coordinated binding between adhesion proteins on adjoining cells stunts cell growth and division to keep cells from piling up on each other and forming clumps or lumps. In cancerous cells with an E-cadherin, contact inhibition is lost as part of the progression to uncontrolled growth and/or proliferation to form tumors and eventual metastasis.

Cells are also able to communicate with cells to which they are not directly connected. Local secretions for example of lactic acid reduce pH and signal other cells of the local meta-bolism. Hungry cells can deplete fuels such as glucose signaling cells more distant from blood supply than the hungry cell perhaps to release factors to encourage angiogenesis, another signaling process. We also have a nervous system that allow peripheral cells to tell the brain what's happening and the brain to tell muscles to get moving. Hormones are another system using a long distance communication. Hormones affect most of our aspects of living including, but not limited to: excitability, sleep-wake cycles, urinary output, urge to eat, glucose availability to cells' mitochondria, blood pressure, puberty, reproduction, growth and development, hair growth, menstrual cycles, fetal development, labor and delivery, lactation, etc.

Life is a complicated interaction of physics (movement—including temperature and sound, light, mass attraction, electricity, magnetism), chemistry (interaction of electrons amongst atoms putting together molecules, etc. and these larger structures, including their functionalities), and biology (melding of chemistry and physics to produce organisms capable of making like organisms [self-replication]). Biology is the science of life, a process embodying sentience, sharing of experiences, remote sensing and transfer of experiences and, as evidenced in humans, ability to understand and improve life itself.

In addition to the intercellular communications discussed above, an ancient form common in single cell organisms as well as in macroorganisms is used throughout our bodies for direct cell-to-cell communications between cells not in direct contact but within up to perhaps five or six cell diameters from one another. One elegant feature of this communication form is the capacity for bulk transference of intracellular contents, including contents as large as intact organelles such as the energy powerhouse, the mitochondrion.

Even in single celled organisms, the molecules in the cells apply physics (movement, temperature, diffusion) and chemistry (making and breaking chemical compounds) to deviate from chemical equilibrium to maintain a living status of dynamic equilibrium. Every cell has to maintain itself separate from its environment while all the while internalizing sustenance from the surrounding environment and using the environment as a waste sink. While evolutionary theory references survival of the fittest, implying competitiveness where the triumphant survives by outcompeting and causing the demise of others, the theory has space for a compromise where part of being “fittest” requires a degree of cooperation with other life forms. The ability to exchange information and benefit from life lessons of other beings can be an important component of being “fittest”. Single cell organisms can communicate through exchanging lessons learned, e.g., an enzyme to use a new food source or detoxify a toxin. The benefit the organism must have a means for intaking, and memorializing the information, perhaps specific folding of a protein that contagiously folds like proteins, perhaps new genetic coding. In simple cells, the membranes delineating the organism from the environment may open to allow exchange of cytoplasmic contents with similar organisms or the information may be transferred using some sort of vector like a plasmid. Once in the simple cell the new material is more or less free to diffuse about since the simple construction of these cells has no internal barricades.

In the more complex (eukaryotic) single cell organisms and larger multi-cell organisms, each cell is subdivided into specialized compartments including, but not limited to: the nucleus for storing and transcribing nucleic acids, the endoplasmic reticulum (ER) for translating nucleic acids into proteins, golgi for processing and packaging the proteins from the ER, mitochondria for providing the chemical energy enabling normal operations of the rest of the cell, etc. The mitochondrion, along with the chloroplast of photosynthesizing plants, is remarkable in that these organelles maintain their own separate store of nucleic acid as a genome that encodes organelle dedicated genetic material that is transcribed and translated within the membranes of the organelle. The mitochondria, like other organelles are similar to unicellular organisms in that they maintain a separate identity space in which they maintain their own dynamic equilibrium while relying on the surrounding cell for sustenance and a waste sink. When mitochondria or other internal component structures lose ability to support the host cell, the cell must i) compensate to readjust and maintain its new dynamic equilibrium, or ii) die. Early mitochondria are believed to have been parasitic organisms inside the host cell, but that over generations of cell divisions the parasite provided beneficial functions to the host while in exchange the host provided a home, substrates and waste removal services in symbiotic relationship with the squatter. This eventually became a mutual symbiosis where both the host and the mitochondrion benefitted each other to the degree that neither can survive without the other.

When cells are part of a large and complex multi-cell organism (macroorganism) similar considerations obtain. No cell can survive without support from many other cells. The organism survives only by relying on each of its cells to coordinate as a unit to guide the complicated interaction of physics, chemistry, and biology, but on a larger scale because each cell must work in concert with its close environment and even some rather distant but cooperating lifeforms, other cells of the organism.

As in single the unicellular organism, each of the cells of the macroorganism applies the same physical and chemical rules to maintain a living status of dynamic equilibrium, but not just for that cell, but for the entire macroorganism. The cells must work with the nearby and distant cells of the organism so that the immediate environment surrounding every cell is conducive to survival of that cell. The cooperation of nearby and distant cells of the macroorganism is indispensable in order to maintain health of the cell and to bolster that cell's ability to reciprocally support its neighbor cells and in doing so, the macroorganism at large. For optimal living, each of the cell's organelles must also be appropriately supplied. Appropriate is a key concept here because no cell and analogously no organelle of any cell can function maximally; each must operate to maintain the dynamic equilibrium. This involves compromises and tradeoffs where no one pathway, organelle, cell, organ, etc. can run rampant to the detriment to the greater good for its macro environment.

For example, the mitochondrion organelle as mentioned above has its own genome. The mitochondrion grows and divides in a process called fission to produce separate progeny mitochondria to support its cell's growth. Mitochondria also join together in a process called fusion to make larger mitochondria. Within the cell a process of mitophagy eats up mitochondria, when situations warrant, to reduce mitochondrial mass and numbers. These processes support the classical duty of mitochondria, to metabolize glucose and supply ATP to its host cell so that the cell can continue its operations. Over many cell divisions, mitochondria have become more simplified and more reliant of the host cell for many of the tools it needs to function. Mitochondria have a sparse genome that is incapable of maintaining the mitochondrial functions. Most proteins of the mitochondrion are provided by the host cell. The minimalistic genome has several advantages. The nuclear DNA is protected by a nuclear membrane, by histones and by robust proofreading that are not present in the mitochondrion. The mitochondrial genome is more easily damaged by physical or chemical assaults and correction is often carried out by destruction of the entire genomic molecule.

In addition to the classical energy supply duty of the mitochondrion, the mitochondrion also is intrinsically involved in cell signaling, cell growth and proliferation, cell metabolism and even cell death. Given these diverse activities of mitochondria, it is not surprising that genetic and/or metabolic alterations in mitochondria appear to be involved in several diseases, including many cancers. Mitochondria as intracellular signaling organelles mediate bidirectional intracellular information transfer: anterograde (from nucleus to mitochondria, endoplasmic reticulum to mitochondria, cytoplasmic membrane to mitochondria, peroxisome to mitochondria, etc.) and retrograde, the reverse. Mitochondria also act as messengers between cells, for example participating in a facet of an offshoot of our juxtacrine signaling system with cytonemes, filopodia, etc. Using these intercellular communication channels, the mitochondrial genome (mtDNA) and even whole mitochondria are mobile and can transfer their intact materials, their functionalities and their stored information to an adjacent cell, especially to one plagued with malfunctioning or nonfunctioning mitochondria.

Mitochondria are specialist movers of electrons, as they oxidize many different molecular species that act as reactants in the cell's biochemical reactions. In the end, O₂ is reduced to form H₂O. In these metabolic processes, mitochondria also produce reactive oxygen species (ROS), powerful oxidizing compounds that may be beneficial when oxidizing an appropriate substrate, but are quite damaging when they react on unintended molecules. A ROS may oxidize a segment of mitochondrial genome to damage the mitochondrial DNA; or may act on a peroxidizable lipid to initiate a damaging peroxidation cascade. When ROS form a free radical that is not attenuated or scavenged, the resulting free radical cascade, a series of spontaneous degradation reactions where an unpaired electron is passed from molecule to molecule, can be extremely damaging to a multiplicity of molecular components in the mitochondrion itself and throughout the cell.

Normally, about 2-5% of the O₂ consumed does not end up as H₂O (water), but is incompletely reduced and becomes an ROS. This results mostly from a small fraction of reducing equivalents diverting off from complex I or complex III of the mitochondrial ETC to form the free radical superoxide anion, O₂⁻. The O₂⁻ itself is needed for some reactions, e.g., for forming iron-porphyrin complexes and for killing invading microbes. The lone electron that provides this diatomic oxygen molecule with its single negative charge causes an unstable configuration resulting from its odd number of electrons—and one electron not having another electron to pair with constantly seeking a pairing partner with other molecules. These molecules with unpaired electron are extremely reactive and can pass the unpaired electron to another molecule in a destabilizing cascade. Mitochondrial superoxide dismutase then converts the O₂⁻ to H₂O₂ (not a free radical), but which can be reduced further to another highly reactive radical OH (hydroxyl radical), a hydroxide ion stripped of an electron.

ROS molecules themselves are important signaling molecules that mediate changes including, but not limited to: cell proliferation, differentiation, transcription, etc. Improper or exuberant ROS activity (sometimes termed oxidative stress), has the ability to initiate lipid peroxidation cascades that will corrupt lipid biomembranes and damage membrane and other intracellular proteins, lipids, and nucleic acids, including DNA. [This is useful when a macroorganism is fighting a pathogen.]

But, the DNA of the mitochondrial genome, itself, is especially susceptible to ROS damage. It is quite close to where ROS is produced (i.e., the mitochondrial inner membrane and the ETC), and the mitochondrial DNA molecule has no introns or protective histones and it has only a limited capacity for DNA repair. Oxidative stress therefore impedes mitochondrial function by directly acting: in the membranes, in the enzyme complexes, in membrane transport; and indirectly: by disruption of mtDNA. Severe or prolonged oxidative stress will lead to irreversible oxidative damage to multiple pseudorandom molecules with consequent cell death. Oxidative stress in mitochondria from functioning during a lifetime of metabolic reactions compromises multiple mechanisms of the original metabolism with the evidence apparent in several aging processes.

The health and function of mitochondria can be improved by, for example, nutrition or chemical supplement targeted to redirect or improve those compromised mitochondria's current status and the current status of the surrounding cells, for example, by providing to the cell and its mitochondria one or more, substrates, cofactors, and/or enzymatic activity modulators including, but not limited to: Riboflavin (B₂), L-creatine, CoQ₁₀, L-arginine , L-carnitine, vitamin C, cyclosporin A, manganese, magnesium, carnosine, vitamin E, resveratrol, α-lipoic acid, folinic acid, fulvic acid, dichloroacetate, succinate, prostaglandins (PG), prostacyclins, thromboxanes, prostanoic acid, 2-arachidonoylglycerol, NSAIDS, melatonin, cocaine, amphetamine, AZT, mitophagic controlling compounds, glutathione, β-carotene and/or other carotenoids, etc. But when mitochondria are severely stressed and nutritional supplementation is insufficient, more robust actions may be necessary for cell survival.

However, some supplements or intended therapeutic compounds taken for immediate benefit will have the opposite effect long term. Some common mitochondrial perturbations that have been recognized to result from ingesting supplements or taking drugs include, but are not limited to: brain swelling, prostate cancer, seizures, heart arrhythmia, cardiac arrest, dizziness, ringing in the ears, reduced absorption of iron, anemia, glaucoma, elevated blood pressure, heart valve and vessel damage, liver damage, confusion, insomnia, drowsiness, renal failure, tendon damage, hallucinations, immune disorders, etc. In many instances, supplements or drugs will: deplete numbers of mitochondria; increase ROS secretions from mitochondria; slow oxidative phosphorylation by mitochondria; decrease ATP production by mitochondria; and/or compromise the integrity of the mitochondrial membrane(s) causing excess heat production.

Antibiotics are especially a risk to mitochondrial health. This should not be unexpected given the similarities shared between mitochondria and bacteria. Antibiotics including, but not limited to: fluoroquinolones, macrolides, clindamycin, chloramphenicol, sodium azide, rifampin, tetracycline, azithromycin, roxithromycin and linezolid inhibit mitochondrial functions.

Bactericidal antibiotics, e.g., quinolones, aminoglycosides and β-lactams (including, but not limited to: penicillin derivatives (penams), cephalosporins (cephems), monobactams, and carbapenems) affect mitochondrial metabolism though interference with the oxidative phosphorylation/ATP production at the mitochondrial inner membrane with resultant increase in ROS release, decreased ATP availability and decreased O₂ consumption. Both mitochondrial and nuclear DNA show antibiotic exposure related damage—with the minimal mitochondrial DNA repair mechanisms compounding coding errors in the organelles.

Quinolones include, but are not limited to: flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, besifloxacin, clinafloxacin, gemifloxacin, sitafloxacin, trovafloxacin, prulifloxacin, etc.

The penicillin family of antibiotics includes, but is not limited to: amoxicillin, ampicillin, bacampicillin, carbenicillin, cloxacillin, dicloxacillin, flucloxacillin, mezlocillin, nafcillin, oxacillin, penicillin g, penicillin V, piperacillin, pivampicillin, pivmecillinam, ticarcillin, etc.

Cephalosporins include, but are not limited to: cephacetrile, cefadroxyl, cefalexin, cephaloglycin, cephalonium, cephaloradine, cephalothin, cephapirin, cefatrizine, cefazaflur, cefazedone, cephazolin, cephradine, cefroxadine, ceftezole, cefaclor, cefonicid, cefproxil, cefuroxime, cefuzonam, cefmetazole, cefotetan, cefoxitin, carbacephems, loracarbef, cephamycins, cefbuperazone, cefmetazole , cefminox, cefotetan , cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, oxacephems, moxalactam, cefclidine, cefepime, cefluprenam, cefoselis, cefozopran, cefpirome, cefquinome, oxacephems, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, nitrocefin, etc.

Monobactams include but are not limited to: aztreonam, tigemonm, nocardicin A, tabtoxin, etc.

Carbapenems include, but are not limited to: imipenem, imipenem/cilastatin, doripenem, meropenem, ertapenem, etc.

Aminoglycosides including, but not limited to: amikacin, gentamicin, kanamycin, neomycin, netilmicin, paromomycin, streptomycin, tobramycin, etc., inhibit mitochondrial protein synthesis by two modes of action. They bind a ribosomal subunit to interfere with peptide elongation. And when tRNA binds the ribosome, an aminoglycoside induced mRNA-tRNA reading mismatch is a frequent occurrence resulting in mis-sequenced, dysfunctional proteins and/or prematurely truncated proteins with no activity or improper activity.

The tetracycline family of antibiotics including, but not limited to: tetracycline chlortetracycline, oxytetracycline, demeclocycline, lymecycline, doxycycline, meclocycline, methacycline, minocycline, sarecycline, tigecycline, rolitetracycline, omadacycline, etc.; as well as the antibiotic chloramphenicol representing another class of antibiotic, preferentially target mitochondrial translation by binding one of the subunits of the mitochondrial ribosome. This extra molecule bound on the ribosome inhibits or prevents amino-acyl tRNA binding to the ribosome or formation of the peptide bond between the amino acids positioned at the mRNA on the ribosome, respectively. The tetracyclines do not exert similar effect on translation of nuclear proteins. The mitochondrial proteins encoded by the nucleus are produced at near standard rates while the few, but important, proteins encoded by the mitochondrial genome are in short supply. The skewed ratio of nuclear sourced proteins to those encoded by the mitochondrial genome creates imbalance and inefficiency in the mitochondrial matrix with a marked decrease in oxidation phosphorylation and a resulting decreased O₂ consumption.

Similar effects are observed in plants indicating that the tetracycline inhibition of mitochondrial translation is one of its basic modes of action. The mitochondria of cells in the presence of a tetracycline were smaller in size and each mitochondrion contained fewer copies of mitochondrial genome per mitochondrion. In such cells, the fusion/fission balance in mitochondrial dynamics is tilted towards fission. Increased mitochondrial fission v. fusion is associated with early stages of apoptosis, a mechanism of programmed self-destruction of the cell. The fission/fusion effect was bimodal—expression of fusion genes in the cell is decreased while expression of fission genes increases. [All these are nuclear genes that encode mitochondrial proteins.]

These antibiotics are merely examples of interventions and reactions that are beneficial to cells when they occur, but that in their beneficial acts leave either transient or permanent artifacts in compromised metabolism of the affected cells, e.g., damaged genomic material, especially in mitochondrial DNA.

Many additional medications and supplements are known to impair metabolism, especially through mitochondrial activities, for example, anticonvulsants such as valproate, affect the cell's well-being and metabolism by sequestering carnitine; decreasing fatty acid oxidation (β-oxidation), Krebs, ETC activity including oxidative phosphorylation and inhibiting complex IV; antidepressants, such as amitriptyline, amoxapine, fluoxetine, citalopram, etc.; antipsychotics such as chlorpromazine, fluphenazine, haloperidol, risperidone; barbiturates such as phenobarbital, secobarbital, butalbital, amobarbital, pentobarbital, etc. that increase the fission/fusion ratio of mitochondria, interfere with complex I activity and inhibit mitochondrial protein synthesis; anxiety relief medications such as alprazolam, diazepam, etc.; anti-cholesterolic medications such as a statin, bile acid, ciprofibrate, etc., by reducing coenzyme Q₁₀ availability, inhibiting the ETC—especially complex I; analgesics such as COX2 inhibitors, aspirin, acetominophen, indomethacin, naproxen, clinoril, diclofenac, etc., by increasing oxidative stress, uncoupling oxidative phosphorylation and inhibiting the ETC; antibiotics such as quinolones, aminoglycosides, β-lactams, etc., by inhibiting β-oxidation, inhibiting mitochondrial protein synthesis; anti-arrhythmics such as amiodarone, etc., by inhibiting β-oxidation; steroids by uncoupling oxidative phosphorylation; anti-viral medications such as interferons, etc., by interfering with mitochondrial transcription; diabetic medications such as metformin, etc., by enhancing glycolysis (diversion to lactate), inhibiting oxidative phosphorylation; β-blockers by causing oxidative stress through ROS; chemotherapeutic medication such as doxorubicine, cis-platinum, tamoxifen, etc., by interfering with mitochondrial transcription; etc. As these perturbations accumulate it becomes increasing advantageous to the organism to have these accumulated “errors” expunged.

In addition to the compounds intentionally introduced into our bodies, competitive biology provides additional impediments to full functioning of our metabolism. Infectious bacteria attack with toxins and other defensive strategies to counter our immune responses. And our microbiome is a shared relationship forcing compromises on our end to support these helpful fungi and bacteria. Our well-being is enhanced by these microbes metabolizing substances to a form we can use and by reducing harmful effects of some of our metabolites. These microorganisms must rapidly adapt to many different substrates as our diets and our metabolic outputs change. One important process microbes use to adapt is: lateral gene transfer (LGT), where fragments of genetic material are exchanged between different organisms.

Bacterial resistance to antibiotics is spread from one species to another by this LGT process. Bacterium-bacterium transfer is estimated to occur by the trillions in our bodies. But concomitant with this interbacterial exchange, prolific release of DNA from bacteria into the environment makes omnipresence of bacterial genetic material a factor that all other organisms must manage. All living organisms on earth, from simple microbes to complex macroorganisms all share the same four nucleotide bases that are polymerized to form their genetic material. Each DNA molecule can be bound to another DNA molecule's terminus as a simple co-polymer addition. DNA translation and repair mechanisms can result in exposed polymer termini and if not protected will allow incorporation of a foreign DNA fragment.

Such foreign DNA has been observed in genomic sequences from somatic human cells. Cancer cells show a higher incidence, possibly because a past insertion in a rapidly dividing cancer cell will be amplified (doubled) with each subsequent cell division, and possibly because the high rate of cell divisions leaves the dividing cells especially accepting to foreign DNA fragments.

All human cells (as well as cells of other organisms) have a large number of retroviral remnants in the nuclear genome, i.e., endogenous retroviruses. These are consistently present in the genomes of most individual in a species. In terminally differentiated (non-dividing) or slowly dividing cells each insertion may only appear in a single cell or a small fraction of cells. The rarity of these insertions is likely to make them appear simply as “noise” or extremely mild contamination in the sequencing data. The inserted DNA is almost certainly much more prevalent than apparent in data collections.

In fact, in a simplified experiment [D. R. Riley et al., “Bacteria-human somatic cell lateral gene transfer is enriched in cancer samples,” PLOS Computational Biology, 2013.], looking at the much smaller and therefore easier to analyze mitochondrial DNA showed that about one third of the samples had detectable bacterial DNA inserted into the mitochondrial genome. In cancer patients, bacterial DNA was about 1000 times more likely to be found in the cancer cells as in the non-proliferating cells. Part of this extreme difference in frequency may be artefactual due to the clonal nature of cancers and their rapid rates of dividing to form new cells, but other factors appear to be involved.

The important message here is that bacterial DNA is inserted into both nuclear and mitochondrial DNA. Depending on the genetic material inserted and its location, (for example: Did it interrupt an important gene or even a critical gene?) the result to the cell's metabolic status may range from: 1. Minimal (when a bacterial genetic fragment is small, is not expressed and if inserted is in a quiescent position in the host cell's genome); 2. Slight (bacterial the fragment a little larger, slightly interferes with duplicating or reading the native genome.); 3. Moderate (bacterial gene is expressed and inhibits or competes with host metabolism; gene insertion alters transcription of a host gene (e.g., up-regulates or down-regulates transcription, or results in an isoform); 4. Extensive (bacterial gene inserts in a position where it regulates oncogenesis or other aberrant growth); 5. Critical (bacterial gene turns on a gene or blocks a gene with results fatal to the cell).

For example, in a sample of acute myeloid leukemia cells, at least a third of the inserted microbial genes were found in the mitochondrial genome. In this case the genes were predominantly from the Acinetobacter genus. In stomach cancers, Pseudomonas appeared to be a preferred LGT donor. In this case though, nuclear DNA was a favored target with five genes especially targeted (four of these five being oncogenes!).

Another example proving the stress on cells from LGT is seen in a 2015 report—Genome Res. 2015 June; 25(6): 814-824—where cancer cell sequences were found to have incorporated the host mtDNA into their nuclear DNA. A recent report from Vinodh Srinivasainagendra et al finds abnormal mtDNA incorporated into the nucleus of adenocarcinoma cells.

Viruses are better known for incorporating and expressing genomic material in host cells. Retroviruses have their RNA reversed transcribed for insertion into the nuclear DNA. Viruses, though, have multiple attack protocols on cells, often affecting mitochondria. For example, many viruses including, but not limited to: hepatitis C, herpes simplex, hepatitis B, Coxackie, Epstein Barr, etc. modulate Ca⁺⁺ flux in mitochondria and often in the endoplasmic reticulum. Such modulations shift balances of numerous metabolic reactions.

Some viral attacks on cell organelles are relatively specific. Hepatitis C guides delivery of the Parkin protein to the outer mitochondrial membrane leading to loss of its integrity with resultant increase in permeability and ROS generation and release. ROS attack leads to ETC reduced complex 1 activity and initiates a bulk autophagic reduction in mitochondrial numbers mass mitophagy). Removal of the damaged organelles is consistent with the survival and maintenance of HepC infected cells.

Herpes simplex 1 and 2 employ a nuclease to destroy mitochondrial DNA and the mitochondrial integrity in infected cells. Epstein Barr (EB) binds mtDNA with a single stranded binding protein inhibiting transcription and thus mitochondrial ETC activity while favoring EB replication.

To maintain genome integrity, infected cells experiencing dysfunction in their genomic expression generally begin a self-destruction process of autophagocytosis called apoptosis. A viral infection will generally trigger a ‘burglar’ alarm scenario in the cell to initiate a series of intracellular pathways that together involve a programmed cell death pathway. Many viruses have incorporated strategies to counter this defense. E.g., baculovirus includes instructions for a caspase inhibitor, p35, that is essential for its pathogenesis. Activation of cellular anti-apoptotic genes is used in some cases to convert a lytic viral infection to a persistent infection. Herpes viruses have stolen genetic material from host cells to encode a viral Bcl-2 analogue of the anti-apoptotic Bcl-2 gene present in most cells.

Post infection many viruses leave latent proteins in various cell membranes that exert lingering effects on functions including, but not limited to: transport, differentiation, kinase activation, mitochondrial fission/fusion protein balance, etc. EB takes advantage of otherwise natural antiviral and cellular defense mechanisms in a manner to promote EB survival in infected B cells. Post infection host genes including ISG20 (interferon stimulated gene), DNAJB2 (DnaJ [Hsp40] homolog and CD99), CDK8 (cyclin-dependent kinase 8), E2F2 (E2F transcription factor 2), CDK8 (cyclin-dependent kinase 8), and ACTN2 (actinin, alpha 2), exhibit lingering effects from the viral attack. Several viruses comprise coding for fragments of our native proteins. Imprecise response of our immune systems can result in autoimmune disease. Similarly, a vaccine, for example from a ruptured pathogenic organism may comprise fragments similar to our native material. These may also instigate an autoimmune response. Inhibiting TNT formation after possible or probable exposure to a virus but before a vaccine will have elicited an antibody response may be useful in responding to an outbreak, epidemic, pandemic or bioweapon attack. The anti-TNT therapy may slow viral cell-to-cell propagation sufficiently to prevent disease for days or up to the two weeks until the immune system has had the time to produce a protective titer.

These events, whether medical intervention, microbial attack, our body's natural defensive response, needed ROS production diverted to an unintended target, artifact of viral infection, etc., comprise a myriad of life events that effect chiefly mitochondria, but can have profound effects throughout the cell. Not all these errors have been characterized sufficiently to allow, for example, a technician to synthesize a specific interfering RNA to be inserted in the cell as a corrective response.

In some cases, scientists have identified one or more genes, organelles or proteins whose activities compromise or are compromised. It then is possible to genetically engineer responsive therapies that address the recognized dysfunction. But this basic strategy suffers at least two problems. First, while a primary defect may be recognized, its sequellae may not be fully appreciated and thus would be untreated. Second, the recognized defect may not be primary, but a result of an upstream defect that is not as obvious, and may exert multiple downstream effects. Third, the defect may not in fact be a true defect, but, in fact, the optimal opportunistic response available to the cell so that eliminating the defect may undo the cell' reactive response and make the situation worse. Fourth, the observed defect may only be the most obvious, leaving multiple defects unaddressed.

Clearly, in the dynamism of life, many things can go wrong. But our species and many others survive to this day. Our cells have adapted to these multiple stresses with responses to minimize or reverse the external or internal assaults. This invention features methods for stimulating, redirecting, or inducing our natural mitigation and repair tools. Or when these repair tools are being used in a detrimental fashion to redirect, slow or shut down the detrimental natural response.

Human cells have developed several robust means to maintain their survival including those that assist their neighbor cells to support the host organism's well-being. Cells assist each other and exchange information by controlling interstitial and circulatory conditions such as providing salts, nutrients, carrier proteins and cargoes, signaling switches, etc. through endocrine, circulatory, paracrine and neural, neuroendocrine, juxtracrine and lesser defined systems. Cells have also developed a mechanism though which one cell acts as a donor and provides one or more of its energy powerhouses to at least one neighbor cell with dysfunctioning mitochondria and/or other anomalies. Facilitating existing mechanisms to correct these errors, improve metabolism, including oxidative phosphorylation, β-oxidation, and other mitochondrial specialized functions, may be used to extend healthy life of organisms including human organisms.

Cell membranes are strong barriers: generally providing passage only to molecules that have, embedded in the cell membrane, a visa molecule (a transport protein that allows a molecule to go across a membrane border) dedicated to that molecule or class of molecules to allow uptake and/or secretion. Different cells will have different visas available in their membranes. Depending on cell type and the transport proteins being expressed in that cell at that instant in time, a molecule will be capable of entering some cell types or cells but not others.

Transport proteins may be quite specific in the molecules that they will recognize and carry across a membrane. Other transport proteins may be less specific, perhaps, for example, transporting molecules with an exposed amino group. And some membrane crossing can be through pores that may have size restrictions or charge restrictions, perhaps a concentrated (+) charge, such as a small ion or a +2 or +3 charged molecule. Other pores may have few limitations, simply providing an aqueous pore through a membrane that for the time that the pore is open, allows rather free passage of any molecule of a size that can flow through.

But there are limitations to what a pore can transport. For example, mitochondria themselves, comprise membranes with their own characteristic transport proteins. Though small in size (˜1-3 μm) in comparison to the host cell (10 s to 100 s of microns in diameter depending on type of cell and stage of development), mitochondria are magnitudes in size larger than the molecules granted passage through gated pores or by carrier proteins. Physical limitations prevent proteins (single nanometer scale in size) from forming transmembrane pores of a size and duration required to pass a substance as large as a mitochondrion. A pore of such magnitude with a diameter several magnitudes larger than the membrane thickness would theoretically cede the cell's control of ion partitioning. Mitochondria are in fact dynamic organelles present in a cell as a network of long, filamentous structures constantly moving along the intracellular highway of the cytoskeleton. Individual mitochondria may elongate or become more rodlike; they may divide in a process called fission into plural mitochondria or nearby mitochondria may fuse together to combine, share and/or exchange or replace mitochondrial components. As a result of their shear size, the cell membrane is an effective barrier to mitochondria and other similar sized organelles passing from cell to cell.

The health of mitochondria in a cell is important for many reasons. As mentioned earlier, damaged cells will undergo a process called apoptosis, a programmed orderly procedure to remove unneeded cells and rather efficiently recycle their materials. An often unappreciated but important function of mitochondria is their function in mediating intrinsic apoptosis, the energy-dependent cell death pathway that occurs in response to physiological or pathological cell stressors, such as toxins, viral infections, hypoxia, hyperthermia, free radicals, and/or DNA damage.

Apoptosis in a cell is induced by the decline in number or activity of anti-apoptotic proteins, (e.g., Bcl-2 and Bcl-XL) and/or by activation of pro-apoptotic proteins (e.g., Bax and Bak). This apoptotic pathway requires Mitochondrial Outer Membrane Permeabilization (MOMP), a critical, irreversible step that commits the cell to cytochrome c liberation and ultimately to a programmed destruction of the cell. As a direct consequence of MOMP, large amounts of cytochrome c along with other apoptogenic proteins are released from the mitochondrial inter-membrane space (space between the two mitochondrial membranes) into the cytosol, where when pro-caspace9 binds cytochrome c, caspase3 activates a cascade leading to: proteolytic cleavage of intracellular proteins, DNA degradation, formation of apoptotic bodies, and other morphological changes that are considered hallmarks of apoptotic cell death. This intrinsic apoptotic pathway is essential for embryogenesis, normal growth and development, tissue remodeling, aging, wound healing, immune response, and for maintaining homeostasis in the adult human body, but when overzealous, the excess cell death is a detriment to the organism's optimal functioning. The apoptotic process is a controlled process minimizing damage to surrounding cells and in fact serving as a recycling mechanism for the cell's component parts.

Every stress to a cell of course does not elicit an apoptotic response. Cells are tolerant of damage and have tools, either inducible or constitutive, that can ameliorate at least some of the damaging effects they encounter. Mitochondria and other organelles have several antioxidant protections, including, but not limited to: superoxide dismutase (SOD), catalase, glutathione and glutathione peroxidase, ascorbic acid, peroxiredoxins, etc. But sometimes the cell is unable on its own to restore adequate function, and absent a rescue of some sort, will suffer an apoptosis induced cell death or less organized necrosis.

A cell under stress first modifies its internal metabolism management to: perhaps substitute glutamine as a fuel source for glucose when glucose supply is limited, glucose is needed for other pathways or enzymatic machinery to turn the glucose to ATP is insufficient, damaged or otherwise employed. Damaged cells though can also request help from their host organism and its cells. For example, they may secrete a chemical request for angiogenesis—bigger, better, closer, blood vessels to supply more nutrients and/or to remove their wastes. They may also reach out to their near neighbor cells for chemical or biologic assistance. One method through which cells of macroorganism can assist each other is to share cytoplasmic contents to restore those lost or lacking in a neighbor cell.

Cells are capable of transference of biochemical, electrophysiological and biomolecular sub-cellular constructs as carriers of material and information through continuous cytoplasmic intercourses between neighboring cells that are opportunistically provided on demand. These connections facilitate intercellular exchanges including, but not limited to: organelles, macromolecules, lipids, ions, etc. from cells able to dispose of said biomaterials, etc. to cells standing to benefit therefrom. Portions of the cells plasma membrane including receptors, pore, transport molecules, lipids, etc. also are transported cell to cell using direct contact connections between their plasma membranes. Proteins, lipids, phospholipids, etc. present in the plasma membrane, especially with presence in the outer bilayer, flows from one cell to another across these intercell membrane connections.

Cells in distress can also request help from or rescue by other cells when the distressed cells secrete or release chemical products of their stressed metabolisms. As a rescue mechanism, many cells have capacity to act as first responders to construct tunneling nanotubes (TNT), a transient structure of a size compatible with in vitro organelle transfer from cell to cell as a rescue facilitator. The primarily distressed cell more often will extend a pro-TNT structure that will come in contact with a recruited rescue cell. TNTs are pipe or tube-shaped, F-actin protein based relatively long-distance cytoplasmic extensions that are several times in length and width longer that the plasma membrane thickness. These extensions may be longer than the initiating cell's diameter. Some, but not all TNT projections contain tubulin or microtubules from the cell's cytoskeleton. When formed, the relatively large diameter inside a TNT does not provide a strong barrier to ions or to small molecules including proteins and thus TNTs have been observed passaging a combination of water, calcium ions, major histo-compatibility complex (MHC), mitochondria, the endosomal-lysosomal system, membrane segments and components, golgi complex, lysosome, ER, prions, and viral and bacterial pathogens between cells.

TNTs provide continuity between the cytoplasms of even non-contiguous neighbor cells allowing transfer of cytoplasmic inclusions including ions, proteins, vesicles, organelles, etc. across multiple cell diameter and even around or bypassing intervening cells. The TNTs are labile structures that form within a few minutes for short length TNTs or may form over several hours when the TNT bridges up to 5 or 6 cell diameters. TNTs may last from just a few minutes up to several hours. They may form rather transient cell-to cell connections in response to a single demand event. TNTs are also dynamic and repeatable capable of establishing a cellular and subcellular network amongst cells that may persist for days and may switch between cells to opportunistically provide the networked cells with optimal resources to meet their constantly shifting requirements.

But TNTs are also capable of being put to use to harm the host organism. The same routes used to carry beneficial nutrients, organelles and helpful cell-to-cell signals can also be turned to spread pathogens and harmful to the host messages between cells.

Typical length of TNTs, e.g., those induced by M-Sec is about 18 μm, commonly between about 10 μm and 30 μm with longer M-sec TNTs (˜40 μm) not uncommon. TNTs may connect pairs of cells via a single TNT or multiple TNTs, but multiple TNTs from one cell usually interconnect several cells thereby forming local networks.

TNTs and TNT like structures have ancient origins. They appear in multicellular animals and plants. Bacteria also form homologous structures between the same and between different species as one means of LGT. The TNT structure might therefore be considered a primordial structure allowing cells to communicate with other cells for mutual benefit or to rescue a distressed cell. This communication process is used by various cell types, and may involve trafficking of many different cargoes between connected cells. TNT-mediated cell-to-cell exchange thereby contributes to cell homeostasis, to spontaneous tissue repair, to the spread of pathologies, and to sharing resistances between cells to therapeutic interventions.

Useful physiologic functions for TNTs include but are not limited to: cell-to-cell transfer of membrane patches, large structures such as membrane vesicles, organelles, electrochemical gradient, ion fluxes like signal transduction molecules (e.g., Ca⁺⁺). For example, myeloid-linage dendritic cells and monocytes transmit calcium signals within seconds through their networked cells connected by TNTs.

TNTs are related to but distinct from filopodia which are exploratory, information gathering, cytoplasmic projections that are made of parallel bundles of F-actin. Filopodia formation relies on an actin nucleation complex anchored by the Rho GTPase CDC42 at the cell membrane. The end of the filopodia F-actin tip has barbed-end proteins, such as capping proteins with Ena-VASP proteins regulating actin polymerization inside the filopod. In contrast, cytonemes and TNTs are thin membrane bridges. Cytonemes are F-actin-containing cytoplasmic projections that transfer surface-associated cargoes from cell to cell. Substances bound to the outside of the membrane can proceed along the cytoneme surface to another cell. Cytoneme formation relies on specific ligand-receptor interaction between the tip of the cytoneme and the target cell. Filopodia and cytonemes are essentially solid structures, not open pipes or conduits. Because the cytoplasms are not in connection between the cells, these interconnections are non-tubular. Cytoneme outgrowth relies on a specific signal gradient triggered by the target cell. TNTs are also F-actin-based but are tubular connections with a biomembrane pipe forming a sealed cytoplasmic path between the separate cells. Thus, cytoplasmic content: ions, proteins, vesicles, organelles and the like can pass through TNTs but not through the other structures. Filopodia, cytonemes and TNTs all though are vehicles that allow membrane components to flow from one cell to another.

Small GTPases are a family of hydrolases with molecular masses usually in the range of about 20-25 kDa. They can bind and hydrolyze guanosine triphosphate (GTP) and function as molecular switches in intracellular signaling to control a wide variety of cellular functions. Members of the Rho family, a subfamily of the Ras superfamily, play a role in actin cytoskeleton organization, membrane traffic, and multiple other cellular functions.

The characterization of TNT-like bridges from several cell types shows differences in cytoskeletal composition and the manner by which they interconnect cells. The differences are evidence that TNTs though categorizable as a general class come in different formations for different functions. But the large number of cell types found in the literature in which TNTs or TNT-like structures have been observed and the ancient origins of these structures shows that TNTs are a general mechanism for functional connectivity between cells.

In addition to ROS and immune secretions as signals that may be used individually or in combination to expedite TNTs, cardiolipin; hemeoxygenase-1; sirtuins; heat shock factors; including active fragments—including, but not limited to: HSP27, HSP40, HSP60, HSP60-HSP10, HSP70, HSP90, HSP110, etc.; heat shock factor 1, including polymers; phosphorylated eukaryotic translation initiation factor 2α; activating transcription factor 6; histone deacetylase, cytochrome c; formylated peptides; intact in interstitium, clumped, polymerized, coordinated with or bound to lipids, carbohydrates other proteins, complexes and fragments of these and similar and analogous chemical signals may also be used to expedite or excite and guide TNT initiation, growth and production. Pharmaceutical interventions are available to facilitate TNT production or to suppress TNT activities. Cannabinoids, including, but not limited to: anandamide (AEA), 2-arachidonoylglycerol (2AG), palmitoylethanolamide, noladin ether, O-arachidonoyl ethanolamine, oleoylethanolamide, plant and/or synthetic cannabinoids, including, but not limited to: cannabigerolic acid, cannabidiolic acid, Δ⁹-tetrahydrocannabinolic acid, cannabichromenenic acid, cannabigerovarinic acid, cannabichromevarinic acid, tetrahydrocanabivarinic acid, cannabidivarinic acid, cannabigerol, Δ⁹-tetrahydrocannabinol, cannabidivarin, cannabichromevarin, cannabichromene, cannabigerivarin, tetrahydrocannabivarin, N-isobutylamides, β-caryophyllene, pristimerin, euphol, N-acylethanolamines, Δ⁸-tetrahydrocannabinol, guineensine, capsaicin, resiniferatoxin, HU-210, HU-331, JWHO15, SATIVEX™ or its generic, dronabinol, nabilone, ajulemic acid, CP 55940, CANNABINORTM or its generic, methanandamide, THC-11-oic acid, TARANABANT™ or its generic, etc. may act in conjunction with sirtuins; HSPs, e.g., HSP90 (a CB₂ chaperone), or through TRPV1 to upregulate HSP27, HSP70, HSP90, etc. Any of these compounds or fragments may be provided intact, as a compound cleavable from a carrier portion, or attached to a carrier portion. One or more methylene groups, —CH₂—, may be incorporated internally within the carbon chains to alter effects including, but not limited to: selectivity ratio for alternate receptors, half-life of activity, rate of delivery, level of activity, toxicity, cost of production, etc.

In several instances, molecular interactions of cannabinoid compounds with effector pathways for potentiating TNT formation are established. In many cases however, the precise pathway remains to be characterized. The pathways and interactions suggested herein relate to current understanding whose knowledge is not essential for practicing the invention. The suggested interactions are incorporated herein for guided understanding and should not be understood as dictum or as stages required for practicing the invention. The skilled artisan will also understand that these interventions are dose dependent. At therapeutic dosages affecting compromised cells with a reduced threshold for eliciting a distress call, less compromised cells will not see their thresholds breached; the needy cells will entreat one or more enabled neighbor cells for rescue through TNTs and/or other means. However, at higher dosages, the intervention(s) prompting the distress signal(s) would be expected to goad comprised cells to undergo actions associated with a poorer outcome, for example signaling their initiation of apoptosis. Signals resulting from these excessive dosages would have no reason to cause a TNT response.

Ascorbic acid (vitamin C) can be used in the presence of activating cannabinoids to potentiate activity of CB₁. H⁺ and high glucose increase TNTs. Growth factors including, but not limited to: EGF, FGF, TGFB-3, etc. and their analogues or active fragments thereof are additional tools that may be delivered to interstitial areas or neighboring cells to accentuate TNT formation. The antibiotic, zeocin, is also usable as a TNT stimulator. Delivery of these activators includes delivering as a portion of a molecule, whose delivery portion may remain attached or be partially or entirely cleaved to present the activity. These activators may be used individually or in combination with other active or carrier substance(s).

Passing mitochondria from one cell to another cell may exert several effects. The passaged mitochondria may replace non-functioning mitochondria of the recipient cell. But even mitochondria that appear to function in energy production and other mitochondrial metabolic tasks may threaten cell survival. Even if the mitochondria of a cell are synthesizing ATP, survival of the cell may be incompatible with its mitochondria. For example, mitochondria are notorious for their epigenetic influence on the host cell's genome, not just for transcription and translation of mitochondrial proteins, but of many non-mitochondrial attached genes that are involved in metabolism. When epigenetic influences are transient or reversible they may be corrected using mitochondrial re-engineering when achieved molecularly by the cell or by mitochondrial transfer between neighbor cells to optimize epigenetic status.

To establish a continuous open-ended TNT connection, membrane fusion needs to occur between the nascent TNT tip and the targeted cell. Since the lipid bilayers have charged hydrophilic/lipophobic surfaces, merging of two lipid bilayers requires disruptive energy to allow continuity of the membranes. This energy can be provided by fusion molecules, such as SNARE proteins or in some pathologic cases, viral fusion proteins. These or alternative membrane-fusion molecules located at the tip of the nascent TNT can be used for breaching the targeted cell's membrane barrier. In some cases, the lipid bilayer composition will favor membrane fusion because, depending on its molecular structure, some lipids adopt a specific curvature that in the right environment, e.g., ionic strength, nearby lipid influences, results in spontaneous membrane fusion. The lipid composition at the end of the TNT when it provides a high degree of curvature will provoke spontaneous membrane fusion between the TNT tip and the targeted cell. Viral fusion proteins can be delivered by vectors engineered to be harmless carriers of the fusion protein of interest without ability to otherwise cause infection.

TNTs can also arise by a process analogous to that of filopodia, that is, with actin involvement in guiding the tubular formation. Actin, when attached to a membrane generally binds through the C-terminus of ezrin, a membrane associated protein, either in a cytoskeletal context or when involved in a protrusion.

TNT initiation and growth can be induced using, for example intact or active fragments of, immune system cytokines and/or mimetics or biosimilars and/or one or more cell distress signal, either induced for endogenous production at the site or added from an extraneous source individually or as part of a combination cocktail. For example, tumor necrosis factor, as an example of one distress signal protein, can be used to initiate outgrowth towards a target cell. Macrophage M-sec protein can also be employed in the initiation and growing process when it interfaces with Ral-A, a filamin binding protein that cross-links actin strands. TNT length is controllable using CD42 which when bound to GDP limits TNT length. In the presence of proper nutrition and lipid supply M-Sec promotes membrane formation thereby providing raw structural material for the TNT tube. In at least one cell class, such as T cells, stimulation of Fas though a Rho GTPase induces TNT formation and elongation. Histone deacetylase inhibitors promote TNT rescue especially promoting TNTs whose intended cargo is mitochondria. Analogous proteins or biosimilars may be used in place of any of these native proteins for the same effect. Fas is one important tunneling nanotubule associated protein whose function has been shown to prevent autoimmune responses.

TNTs may also appear in proximity to filopodia. The filopodic connection establishes a linkage holding the cells in proximity to one another which, because of the reduced distance, results in a lowered initiation threshold necessary to carry out the growth and maintenance of the TNT. TNTs may result after filopodia form and then retract leaving the TNT connection between the cells.

Hydrogen peroxide (and several other oxidative stressors) act through p53 initiated pathways cascading to form TNTs. This mechanism may on rare occasions allow emptying of severely stressed cells, but generally is a process whereby recoverable cells reach out to neighbors to obtain undamaged repair components. The stressed cell reaches out to a neighbor cell by producing a gradient, e.g., through release of H⁺, H₂O₂ or another cytoactivator. The responding neighbor donor cell then initiates TNT growth which continues along the signal gradient to the stressed recipient cell. The TNT apex then merges with the stressed cell's plasma membrane to form a continuous passage through which ions, biomolecules and/or organelles can enter the recipient cell to effect its recovery. One or more gradients including, but not limited to: a temperature gradient, an electrochemical gradient, an osmotic gradient, an ionic strength gradient, a pressure gradient, a magnetic gradient and/or an electric field gradient can be exogenously initiated or applied to drive TNT formation. Since p53 is an essential component of the TNT pathway, blocking or inhibiting p53 will block or inhibit TNT initiation and prevent passage of the macro substances between cells. p53 function is eliminated by siRNAs, either dominant negative. Small molecule inhibitors of p53 include but are not limited to: pifithrin-α/(PFTα) HBr, reACp53, pifithrin-μ, NSC348884, etc. Pifithrin-a inhibits p53 activity by inhibiting p53-dependent transactivation of p53-responsive genes. ReACp53 is a cell-penetrating peptide, that inhibits p53 amyloid formation. Pifithrin-μ inhibits p53 reducing its affinity for Bcl-xL and Bcl-2. Pifithrin-μ also inhibits HSP70 function and autophagy. NSC348884inibits the p53 pathway as a nucleophosmin inhibitor, inhibits cell proliferation, and induces apoptosis in a concentration dependent manner in various cells.

Stress is a strong stimulant for TNT formation. For example, malignant cells under ischemic stress release exosomes that stimulate TNT formation. Inflammation in general promotes TNT initiation and growth. TNTs are important promoters of healing at the margins of wounds. Post ischemic recovery in cardiac and central nervous system tissues involves proliferation of TNTs sharing healthy cell components with nearby damaged cells. While TNTs are integral to repairing, redirecting and rebalancing efforts in macroorganisms they do not act alone. Another messenger system that carries small information bearing or corrective molecules within an active range including the ranges in which TNTs operate has as part of its functions stimulating TNTs. Cell membranes, especially membranes of stressed cells bud off small vesicles approximately 1/100 the size of a red blood cell. Since these exosomes are spawned by a cell's plasma membrane, they comprise molecular constituents of their cell of origin which includes membrane lipids, proteins, and often RNA, including mRNAs and/or miRNAs. The exosomal protein composition is determined by the originating cell and so composition analysis can determine cell and tissue of origin. In addition most exosomes contain an evolutionarily-conserved common set of protein molecules. The protein content of a single exosome, ranges up to about 20,000 molecules and many will generally include all or several of these proteins in addition to their more cell specific cargo: HSPA8, CD9, GAPDH, ACTB, CD63, CD81, ANXA2, ENO1, HSP9OAA1, EEF1A1, PKM2, AGO2, YWHAE, SDCBP, PDCD6IP, ALB, YWHAZ, EEF2, ACTG1, LDHA, HSP90AB1, ALDOA, MSN, ANXAS, PGK1 and CFL1.

A macroorganism's symptoms can be used as a starting point for selecting strategies for rebalancing metabolism through improved intercellular communication. Biological assays can focus on particular tissues and/or blood samples, breath samples, urine samples, hair samples, stool samples, etc. may be analyzed to more specifically target selection and delivery of therapeutic intervention to the recipient. Surveys and/or questionnaires can also be used for guiding selection. These or modified assessments are also appropriate tools for judging effects and can guide modifications as the macroorganism responds to therapy.

For example, data can be collected at multiple levels to improve therapeutic effectiveness. Group or population surveys or averages can be used to suggest most efficient targets and delivery strategies. Data can be collected from the same source over a plurality of time courses to monitor changes with time and rates of these changes. “Big data” and artificial intelligence may be useful for identifying and validating available and more lucrative rebalancing targets and for evaluating effects of practices used for rebalancing. Algorithms developed using the data may be specific to a cell type, an organ or tissue, an individual or to any defined group of individuals

One option available for practicing this invention applies nano sensing technology, either non-invasively, for example, by sensing breath, urine, etc., or by using nano probes given a physical presence within an organism or in one or more selected locations within the organism. A selected location may be in the vicinity of any tissue or perhaps a suspected tumor. The sensor location might be at a site distal to a major target of therapy, perhaps where a metabolite of the stressed cell would be further metabolized: for example, liver, kidney, or simply in a blood vessel. Sensing of metabolic outputs such as chemical products and heat are two important applications of this nano sensing technology and its application to arresting abnormal metabolism and the cells responsible therefor.

Data can also be collected internally, for example by sectional imaging or by concentrating on a particular tissue or organ. Imaging may use non-invasive techniques which may include supplemented marker compounds to accentuate particular aspects. Internal collection may involve tissue biopsy where one or more tissues samples are removed for analysis. Analytical devices may be inserted into the body. These may be markers that would indicate specific areas (tissues) with high concentrations or a target of that marker or perhaps high activity of an enzyme metabolizing the sensor molecule. Small electronic sensors either wired or wireless may be used to collect data. These sensors may be placed within the body or proximal to the body in a position where adequate data is accessible. Sensors may take advantage of nanoscale technology to allow their passage through circulation and deposition at a targeted site. The sensors may also be couriers and deliver rebalancing material(s) to specific target sites, for example when metabolic switching is more severe in one body segment or in a specific cell type or cell with high levels of expression of a surface marker. Sensors may be designed to be chemically, electrically, and/or physically sensitive.

These nano sensors ideally will sense presence of signs that are not casually observable. For example, minor or localized temperature variations possibly including their minor metabolic effects, nano signatures in for example, blood, urine, sweat or breath. A receptor that no longer binds to or a receptor that no longer responds to an extracellular signal may be one type of nano event. A receptor that remains in a permanent activation state, perhaps due to its failure to release its ligand intracellularly or extracellularly may shift the cells metabolism or in a more extreme event, for example when the receptor constitutively activates transcription factors synthesis functions can become almost immeasurable. Nano sensors can carry external binding ligands to remain in such target sites.

One special nano sensor has been carried in our bodies throughout our lives. Our microbiome has adapted to the changes our changed metabolisms have served it. Different species or families of organisms within our microbiomes will have adapted their metabolic reactions just as we will have. The microbiome however will also have faced tremendous changes from its predecessors just a century or two back. Bathing and use of body creams, antiperspirants, etc. have constituted major changes in our dermal biome's characteristic environment. Similar changes have wreaked havoc on conditions our various gut microbiomes will have to adapt to.

Cells of our microbiome are semi-independent organisms associated with diverse regions, organs or tissues of our bodies. By harvesting and analyzing various subgroup in our microbiomes (e.g., collecting: stool, blood, saliva, mucus, sweat, dead dermis, deeper dermis, tissue scrapings, etc.) the adaptations of these microbiota will be a window into the adapted systems the host organism has presented to them. The enzymes and other proteins active in various microbes can help elucidate how the host cells in their source regions have progressively adapted their metabolisms. Assaying proteins or reactions of the microbes' proteins can indicate to some degree the source of the microbe and the environment, including for example, an acidic environment rich in lactate, the microbe has adapted to.

Another assessment of the microbial cells would be to sequence individual or collective microbial genomes. Two tracks of analysis might be selected. One would be to use the microbial genes in their adapted, mutated or gene swapped in state as a window to the adapted host metabolism. A second track would be to analyze the microbes for their contributions to the local environment of the host body portion and where warranted seed the microbiome with microbes that can assist in rebalancing the host organism's metabolism in one or in a collection of locales, including microbial intervention that my affect a majority or even almost all cells of the host.

Microbiome cells can be used as sensors to assess near instantaneous metabolic events and status and they may be selected or engineered to help rebalance metabolic paths in the cells which provide the microbe's metabolic turf.

Electronic analogue or digital sensors may be used. Size is not a constraint, especially with regard to extracorporeal components. Micro or nano scale devices are especially preferred. For example, nano probes or nanoparticles can be configured with nano-sensor capabilities. These nano-sensors can be supplied in the vicinity of a tumor or may be applied more systemically, such as in blood or lymph vessels. One species of particle we can make has a form of nano-motor, or means of moving itself. These can be random or can be configured to be thermotaxic (move towards or away from a heat source) or chemotaxic (move along a chemical gradient, such as a pH gradient). Phototaxic (responsive to light-electromagnetic radiation, radio waves) sensors are another example, but these would be effective only close to the skin or other location penetrated by light or as secondary sensors responsive to a primary sensor that directs the secondary sensor to an identified location. Nanoparticles can also be configured as receivers of electromagnetic radiation. Nanoparticles compartmentalized for example by physical and/or chemical means can be queried to confirm location and if desired about the particle's surroundings. For example, the particle may report back an indication of temperature, pH, and or other parameter programmed into the sensor. When the sensor is configured as an antenna, electromagnetic energy can be converted to heat energy at the target location. Nano sensors can be configured to be responsive to selected biomolecules, such as cell receptors, metabolites, markers on exosomes, etc.

A sensor nanoparticle may also be a reporter nanoparticle, a courier nanoparticle and/or a signal nanoparticle able to deliver a preprogrammed substance or to recruit other couriers for delivery when a preprogrammed event is reported. They may be used to monitor progress in therapy and/or to signal timing for subsequent therapies. Nanoparticles can be mostly physical in their action, may include chemical elements to aid in sensing or for delivery and may even transport biologic cargo(es) depending on the whims of the nanoparticles creator(s).

Less technological applications of the invention are also available. Chemicals, especially lipid compositions are heat responsive. Many chemical reactions are temperature dependent. Thermo-dependence is even more evident in enzymatic reactions where subtle temperature changes can induce profound changes in a protein's (enzymes are most often proteins) or RNA's (some RNAs (ribonucleic acids) have enzymatic or binding/presenting activities) folding (3-dimensional structure) and activity.

Nano sensor devices may be targeted at or inserted into stressed areas in the body. Metabolic imbalance is one common stress requiring action by surrounding cells or larger systems of the organism. One response to stress comes in the form of exosomes, tiny packets of cell material released by the plasma membrane to the interstitium, including some that are present in the circulation. Exosomes can be assayed to indicate levels and areas or stress. The site of exosome release can be used as an area of interest for additional nano sensing.

Exosomes cooperate with TNTs as mediators of cell-to-cell signaling through the transfer of molecules such as mRNAs, microRNAs, and proteins between cells. Exosomes released by cancer cells especially during and after chemotherapeutic or radiation damage carry several cancerous mRNAs and miRNAs along with cytoplasmic and membrane proteins. They also recruit TNTs to provide remedial services, such as healthy mitochondria, to help restore health in the cancer cell. In these manners, exosomes from the onco-cells act as regulators of cancer progression.

In general exosomes release is induced by stress in cells. Depolarization, increasing calcium, heat, especially heat fluctuations, binding and activation receptors on specialized cells are general stimulants of exosome release. Clathrin adaptor AP3 and the v-SNARE TI-VAMP (tetanus neurotoxin-insensitive vesicle-associated membrane protein or VAMP7) are active in lysosome secretion.

LGT between cells induces exogenous gene expression and may mediate RNA silencing. Mouse exosomes are internalized and processed by human mast cells (MCs) to express mouse RNA. The opposite occurs using human exosomes and mouse MCs. Analysis of human MC exosomes found ˜1300 mRNAs and 121 microRNAs (miRNAs) but no DNA or RNA.

MiRNAs control protein expression in cells by modulating mRNA activity. MiRNAs therefore have valuable uses in medical treatments. They are short single-stranded (19-25 nucleotides in length) non-protein-coding RNA transcripts that are produced in the nucleus and transported into the cytoplasm. By base pairing to a complementary sequence of a target mRNA they decreases the expression of the target mRNA.

Exosomes are formed by inward budding into large multivesicular bodies in the cytosol. Exosomes are concentrated carriers of genetic and proteomic information as they participate in intercellular communication. Their messages can be received in several ways. 1) Binding to membrane receptors on a target cell. 2) Sharing surface receptors from one cell to another by vesicle-membrane fusion. 3) Merger with the target cell and delivering the contents (protein, mRNA, miRNA). The cannabinoid, AEA, has been observed in some of the extracellular floating membranes on an external surface where it can bind the plasma membrane CB₁ receptors.

MSCs when stimulated by exosomes released from stressed cells amplify the stress response by releasing their own exosomes, but participate in direct healing though initiation of TNTs to remediate the cells signaling stress conditions. Cultured cells demonstrate that the MSC exosomes, not just their mitochondria, have healing properties. Exosomes thus serve two distinct functions in response to stress. They act as messengers to plead for help from other cells. Other cells then provide exosomes that have a remediative effect. And some of these rescue cells are also proximal enough to help the stressed cells through TNTs.

Exosomes are rather simple constructs. Essentially, they are lipid bubbles that may have lipoprotein in the membranes and can carry nucleic acid, proteins, ions and cofactors within the bubble. Partially synthetic exosomes are thus readily obtainable using membranes from selected lysed cells and creating vesicles in media compromising the proteins, RNA, sugars, cofactors, ions, etc. to be delivered to the target cell. The target cell can be refined by choosing the plasma donating cell expressing desired membrane proteins or the proteins can be added during vesiculation. The contents may be selected to contain inhibitory proteins, kinases, mRNAs, miRNAs and/or siRNAs as desired to turn on/off and/or up or down regulated one or more metabolic pathways.

Artificial (partially synthetic) exosomes are not limited to natural compounds. The membrane can be engineered to carry selected ligands to precisely interact with only select cells that bind that ligand. The ligand can be a peptide or modified peptide; the ligand may be a small molecule adapted for display on the exosome membrane. The contents can be inhibitory of select processes, toxic to one or more processes, toxic to the cell and/or excitatory to one or more processes. We therefore can use exosomes to deliver most anticipated smaller molecules or complexes to selected cells.

When used in the presence of or in conjunction with TNT therapy, the combination has extensive and broad uses. Exosome production may be stimulated to initiate TNT production. Artificial or partially synthetic exosomes may be used as an initiator to stimulate exosome release from specifically targeted cells. The exosome intervention may be used to salvage stressed cells or cells about to undergo stress. Select cell types may be thus primed for tolerance to a potentially damaging therapeutic dose. Exosomes may be used to stimulate TNT facilitated wound healing. These are just a few of the many ways TNTs in conjunction with exosomes have special benefits.

On the other side, exosomes might be used to shut down TNTs when TNTs are at elevated risk of damaging the macroorganism. For example, during or following chemotherapy or after exposure to virus, exosomes can be engineered to deliver one or more inhibitors of TNT formation and/or inhibitory RNAs to limit the TNTs contributions to e.g., restoring vitality to a cell damaged by chemotherapy or closing out viruses from intercellular passaging to expand the viral infection.

A major function of TNTs is to provide connections forming a network of multiple cells such that when one cell is stimulated and its cytoplasmic Ca⁺⁺ increases, this Ca⁺⁺ activation is rapidly spread throughout the network in a process somewhat akin to a neural network but without neurotransmitter involved for the cell to cell activation. Heat, pH, hypoxia, and/or chemical and/or biochemical signaling agents may be advantageously applied in isolation or combination to expedite exchange between cells. Intercellular feedback may cause TNT switching events follow a harmonic cycle.

The direct connection that TNTs provide between cells allows electrical propagation directly from one cell to another absent a synapse as used for cell-to-cell information transfer in the neural system. This direct electrical connection aids in connecting cells at the leading edge of a healing wound; and also can be used to repair metabolically compromised cells surrounding or surrounded by healthy cells. Healthy cells may be cells native to the organism and originally at that location. They may be cells native to the organism but driven using one or more chemotactic factor to the region to be healed. They may be cells native to the organism, but removed and cultured in growth or restorative media before return to the organism to aid healing. Or the cells may be immunologically compatible cells cultured from another source and provided as an aid to healing. The healing cells with their direct electrical connections may exert their effects by activating enzymes, such as voltage-sensitive phosphatase, PI3K and protein kinase A.

The direct electrical connection through TNTs allows a population of cells to act as a biological swarm computing system with gating strength determined by cell location, number of connections to a neighbor cell, number of cells connected to, spatial position (determining timing and additive effects), size and content of TNTs, etc. Although less rapid material flux between these connected cells is available to act as a reprogramming mechanism, for example, providing organelles, enzymes, substrate, transcription factors, epigenetic modifiers etc. Even in cases when a transcription factor may have been epigenetically modified in one cell, downstream effect is restorable through these TNTs.

In circumstances where a cell is comparatively worse off and a candidate for autophagocytosis or better positioned than neighbor cells for disposing wastes, the TNTs are used for discarding non-functioning or otherwise disposable biomaterials. This is the opposite of the restorative effect with first responder helper cells that deliver prime pieces to the needy cells. In this arrangement, a cell that may have signaled initiation of apoptosis allows neighboring cells to advantageously “dump” excess, outdated, or waste materials to the cell about to undergo apoptosis. This allows for efficient disposal of wastes and improved functioning of many cells with only the death of the one cell. A variation of this action, such as for intestinal cells when cells at or near the lumen are better positioned to dispose of wastes, involves at least two cells but preferably a network of cells that periodically form TNT connections to deliver their discards to the cell best positioned to eliminate them. The TNTs may form under diurnal control and be inducible by controlling adenosine or other fluctuating metabolites or hormones. Fluctuations, including fluctuations in number, size, flow direction, materials exchanged, etc. may be under a cyclic, (e.g., meal based, diurnal, lunar, weekly) control, may be harmonic relying on a cycling stimulus or simple or multi-stage feedback loop arrangement.

The TNT mechanism can be used to deliver specially selected or engineered mitochondria or mitochondrial like vesicular components to aged, diseased or otherwise compromised cells. Donor cells may be cultured for robust health including a supply of optimally functioning mitochondria or may be engineered to comprise mitochondria or mitochondrial-like vesicles. Mesenchymal or other cells can be modified to comprise such engineered mitochondria or vesicles. The engineering may involve nuclear engineering to provide engineered mitochondrial proteins, may involve mitochondrial or vesicular insertion and/or may involve engineered mitochondrial genome. Even when these engineered donations are transitory, the healing effect may produce durable desired results. The donor cell and mitochondrial material can be specially chosen for a transitory survival or toxic switch characteristic to reduce the risk of or to have ability to switch off future long-term actions.

Cells in a compromised metabolic status with increased lactate release, lead to an environment where interstitial pH is reduced. The increased H⁺ ion concentration is one available signal for inducing TNT formation. An acidic environment thus may be used to augment wound healing and/or to increase intercellular repair (and/or replacement) processes.

Artificial TNT structures have been constructed from biopolymers that are biodegradable. See e.g., biodegradable polymersomes comprising poly(ethylene glycol)-b-poly(d,l-lactide) (PEG-PDLLA) And Meng F.; Hiemstra C.; Engbers G. H. M.; Feijen J. Macromolecules 2003, 36, 3004-300610.1021/ma034040+; Samarajeewa S.; Shrestha R.; Li Y.; Wooley K. L. J. Am. Chem. Soc. 2012, 134, 1235.10.1021/ja2095602. [PubMed]; Fukushima K.; Feijoo J. L.; Yang M.-C. Eur. Polym. J. 2013, 49, 706.10.1016/j.eurpolymj.2012.12.011.

These synthetic TNT scan spontaneously attach to cells and avoid necessity for the original distress signal from the stressed cell or cell-cell initial contact. Slight changes in salt concentrations or osmotic forces exert profound stabilizing or destabilizing effects on these artificial TNTs. Similar interventions have been shown to act on native TNTs. The present invention uses such intervention to either encourage or dissuade cell-to-cell communication, especially to slow or stop exchange of pathogenic organisms and/or pathogenic proteins.

Although TNTs are available for their healing qualities and predominantly used be organisms for beneficial purposes, the TNT mechanism might also be employed to the organism's detriment. For example, if TNTs are induced during a cancer therapy, it is possible that therapeutic effectiveness could be reduced since increased TNT activity might aid healing and maintenance of the cancer cells undergoing chemo-attack. In such instances, promotion and inhibition of TNTs are preferably coordinated with other therapeutic interventions, including inhibited TNT activity during chemotherapeutic, radiation or other cancer cell damaging events.

In addition to the concern that TNTs may support cancer cells in preference to healthy cells of the organism is a concern now with growing evidence that TNTs may mediate the intercellular spread of a diverse group of pathogens. This process may be widespread across life with origination possibly before the separation of animals and plants. There are strong similarities with plant tissues, where cells are connected via membranous channels, called plasmodesmata. This lengthy developmental timeline may have provided pathogens opportunities to turn the TNT from an organism's salvation to a tool aiding their attack.

Formation of TNTs appears to be controlled by several factors including, but not limited to: serum when in culture, glucose concentrations, viral infection, exposure to therapeutic agents, etc. Depending on the patient, the malady under treatment and the cells of concern, the therapist has options for modulating and guiding TNT production, inhibition and/or activity for example by manipulating nutrients; controlling infection or immune response—including using non-pathogenic means and/or killed or attenuated pathogens; vaccines; temperature; pharmaceuticals, including e.g., antibiotics like those mentioned above; hydration; etc.

For example, in vitro, low-serum, low pH, and elevated-glucose stimulates TNT formation in a plurality of cell lines. The same effects can be seen in malignant cell preparations where mitochondrial trafficking from normal mesothelial cells to the malignant cells increased with increased TNTs. O-GIcNAcylation by the O-GlcNAc transferase (OGT) can be used to decrease mitochondrial trafficking and hinder malignant cells recoveries from chemotherapy or radiation damage. Similarly, extreme glucose deprivation or a keto diet, shifting metabolism of all cells, not just the cancerous cells, towards favoring glutamine metabolism over glucose is available to decrease TNT-mediated mitochondrial transfer.

Other stresses, e.g., pathogenic stress caused, for instance, by HIV-infection in human macrophages can increase the amount of TNTs formed between pairs of or multiple macrophages. And chemotherapeutic agents can also influence TNT formation and their cargo trafficking. Zeocin stresses cells by breaking their DNA molecules. Zeocin treatment can be used to increase the number of TNTs formed. While the formation of TNTs may be a counter response that favors cancer cells over the organism, in the absence of cancer, an increased TNT level initiated by zeocin can be used to correct metabolic flaws that have developed in other cells, for example, in aged cells, or cells compromised by a non-malignant disease.

Similarly, cytarabine (ARA), another chemotherapeutic agent, increases intercourse between AML cells and MSCs which donate mitochondria to the AML cells. Topoisomerase II inhibitor, etoposide, and doxorubicin have similar effects. Additional stimulants including, but not limited to: activation by a member of the TNF family, e.g., CD40L, agents that enhance expression of p53, etc.

Mesenchymal Stem Cells (MSC) are active in instructing other cells' functions with small amounts of secreted substances including, but not limited to: growth factors, chemokines, cytokines, bioactive lipids, nucleotides, etc. MSCs also connect to target cells using tunneling nanotubes to transfer the mitochondria and other intracellular components. Target cells can be cancer cells. But MSCs also target other cell types including, but not limited to: cardiomyocytes, endothelial cells, pulmonary alveolar epithelial cells, renal tubular cells, etc., with modifications to these cells' functional properties.

MSCs form connexin 43-containing gap junctional channels with the target cell. It is understood that the physical juxtaposition between MSCs and the target cell through these gap junctions is an early stage of the TNT formation. Ca⁺⁺ at the gap junction then proceeds to help form the TNTs.

As an option to using TNTs the practitioner may use other tools, e.g., mitochondria can be delivered from MSCs to the other cells by microvesicles. Arrestin domain-containing protein 1-mediated microvesicles (ARMMs) can be used to carry MSC mitochondria these microvesicles into phagocytes like macrophages, to increase the macrophage mitochondrial bioenergetics. This also decreases the MSC load of depolarized mitochondria to benefit MSCs. Astrocytes also use vesicles for mitochondrial delivery to neurons. Similar vesicles can be made to take advantage of the neuronal practice of incorporating these corrective boluses.

As an alternative to induced TNT rescue, naked mitochondria can also be fed to and internalized by cells. For example, ischemically, osmotically, nutritionally, chemically or otherwise stressed cells can receive restorative therapy using isolated mitochondria. Macropinocytosis processes by cells in need then engulf and internalize the healthy mitochondria. Endothelial cells are easily accessed through the bloodstream. Lung cells are treated using a mist like suspension of bioparticles in moist vapor. Extra-circulatory infusion of mitochondrial laden liquid can be used for treating other cells. Delivery can be improved when desired by loosening cell adhesion. Osmotic forces may be used to open spaces between cells or to tighten contact. In some areas infusion of additional fluid or gases may act to space out the cells. Injection providing healthy naked mitochondria to a compromised area will restore at least the marginal cells (cells at the margins of the injection site(s)). Injecting healthy mitochondria to older cardiac muscle can restore ion pumping maximums, stronger transmembrane electrochemical potential and improved contractility and rates of contraction and recovery.

Injection of autologous mitochondria, inhibition of cell adhesion proteins using synthetic or even natural components including, but not limited to: PAPC and the noncanonical Wnt ligands, Wnt-11 and Wnt-5a, transmitochondrial cybrids and photothermal nanoblades are additional effective means for dispensing restored mitochondrial functions to cells in need. Not all cells receiving benefit need to be in contact with the mitochondrial infusion liquid or mist, since cells of similar functions are quite prone (more prone than unlike cells that aren't stem or dedifferentiated cells) to form and use TNTs which will distribute or share the restorative benefits.

Lipopolysaccharide induced injuries make tissues more leaky and cells more receptive for uptake of donated naked mitochondria or through MSC or other available cell intercourse. Rotenone and components of tobacco smoke also can be used to induce mitochondrial transfer and other TNT vectored exchanges.

MSC mediated TNT exchanges are sometimes detrimental to the organism. MSC donation to adenocarcinoma cells has been documented with the resulting recovery of the mitochondrial function (including O₂ consumption) in these cells. These exchanges also increased concentrations of both the mitochondrial DNA and amount of ATP produced. In these and other cancer cells MSCs or other mitochondrial donors have restored some normalcy to cancer cells as evidenced by both basal and maximal O₂ consumption, reduced glycolysis, reduced lactate and increased ATP availability. In some, but not all cases, improved mitochondrial integrity correlates with increased rates of proliferation. Thus, treatment of these cancers from the point of view of the macroorganism is enhanced by slowing or halting TNT formation rather by encouraging it to make healthier cells. These mitochondrial restoration effects that might ordinarily be considered beneficial, e.g., when restoring aged tissues, organs, cells, etc., overcoming stresses on tissues, or repairing otherwise damaged tissues, become detrimental to the macroorganism, when the cellular restorations facilitate acquired resistance to therapies or spare apoptosis of unneeded cells. The skilled artisan will investigate the situations inherent in the proposed recipient and determine, when, where and under what conditions TNTs should be encouraged or inhibited. The knowledge that mitochondria, other organelles, membrane components, etc. can be exchanged, transferred or donated spontaneously between cells and/or that naked mitochondria locally infused or otherwise provided are available methods for restoring or optimizing cell functions will allow improved treatments and therapies for aging, diseased, damaged and/or simply suboptimal cells for any reason.

One concept to understand is that once even a small number of cells have restored health, MSC participation is no longer necessary. Healthy cells in contact with other cells not so blessed will form nanotubes and transfer mitochondria and other cellular components, including proteins, Ca⁺⁺ and membrane lipids and lipoproteins to the beneficiary cells.

Several pathogens have the wherewithal to employ TNTs for cell-cell transmission of disease. Human immunodeficiency virus type I (HIV-1) avoids contact with circulating antibodies by spreading from an infected cell to and uninfected cells through TNTs. Protein based disease such as self-propagating aggregated isoform of prion protein PrPSc, and cytotoxic amyloid beta, exploit TNTs for their intercellular route to spread the disease. TNT events are also associated with intercellular transfer of P-glycoprotein, a transmembrane transporter protein that pumps many chemotherapeutic drugs out of cells. The transfer of P-glycoprotein from non-tumor cells to tumor cells followed by tumor cell to tumor cell sharing allows every tumor cell to eventually gain multi-drug resistance with the few that never receive the pump being replaced by the defensive cells.

Inflammation is one method for inducing TNT formation. Gram-negative bacteria expressing the endotoxin lipopolysaccharide (LPS) are known inducers of acute inflammatory responses in mammals. The frequency of MHC class II cell connections to other cells increases in response to infection and to LPS stimulation in experimental situations. Similarly, TNT-like structures are formed between MSCs and LPS-injured lung alveolar cells, and mitochondrial transfer from MSCs through these structures contributes to tissue repair.

According to Loai K. E. A. Abdelmohsen et al, J Am Chem Soc. 2016 Aug. 3; 138(30): 9353-9356, biodegradable PEG-PDLLA nanotubes can be synthesized in the presence of between about 2 to 5 wt % of hydrophobic doxorubicin (DOX). PEG-PDLLA copolymers will spontaneously assemble into spherical polymersomes. Such biodegradable polymersomes can then transform into nanotubes when dialyzed under hypertonic conditions, with increasing [NaCl] leading to both structural enrichment and elongation. Such well-defined nanoparticles are available for practicing this invention where nanoscopic control over size and shape is considered important or relevant.

The influenza virus A (IAV), which afflicts birds and mammals, enters the host cell via receptor-mediated endocytosis wherethrough viral particles gain initial entry to cells of a macroorganism by binding to a cell surface molecule containing sialic acid. To gain entry bind to the cell a viral particle must evade the immune defenses present in the circulation or mucous if entry is via that path, then migrate to a cell with a specific protein complementary to the binding protein on the viral surface, bind the cell and become internalized before being recognized as an alien dollop of nucleic acid. The particles become internalized within clathrin-coated pits as they are transported to endosomes. Within the cell endosome, the acidic environment activates the M2 ion channel selectively transport H⁺ and K⁺ into the virion interior and there to dissociate the M1 protein from the ribonucleoproten (RNP). Low pH (pH 5.0 to pH 5.8, depending on the HA subtype) triggers an HA protein refolding event merging of the viral membrane with the endosomal membrane, and to release the RNPs into the cytoplasm for transport to the nucleus, for viral transcription and replication. To assemble virions, RNPs exit the nucleus through nuclear pores and must be carried by cytoskeletal microtubules to the plasma membrane. There M1 interacts with the viral RNP and in conjunction with HA, NA, and M2 assembles into viral particles at the plasma membrane. In polarized cells, budding occurs from lipid rafts on the apical surface, which are highly enriched in cholesterol and contain large amounts of glycerophospholipids and sphingolipids.

Neutralization of the invading virus in the circulation with antibodies induced either by prior infection or vaccination is a primary mechanism to prevent flu. However, even in the presence of circulating protective levels of antibodies, influenza viruses can still infect and spread to cause disease. The flu virus is able to simplify its packaging and avoid the hazardous extracellular immune system by inducing and then passaging through TNTs connecting the infected cell to neighbor cells. And these neighbor cells once partially commandeered y the viral nucleic acid segments likewise transmit more viral nucleic acid to other cells. The collection of near neighbor cells infecting a significant population of neighbor cells can by creating a population of infectious cells so to speak under the immune system radar can relatively easily produce a number of viral particles that when released by the now numerous sub rosa infected cells are of sufficient quantities that many will escape immune attack and spread the infection far from the site of initial infection.

This process is not beholden to influenza. Another well-known virus, HIV-1 (Human Immunodeficiency Virus 1) makes use of the body's TNT system to maintain its infective strength. HIV-1 infects vital CD4 T cells in the human immune system along with immune cells such as Macrophages and dendritic cells. Infection with HIV-1 causes progressive loss of CD4.

T cells, leading to the severe immunodeficiency giving the disease its name, acquired immune deficiency syndrome, (AIDS). We now understand that infection continues in the absence of the CD4 positive cells through TNTs that physically connect HIV-1-infected [CD4+ cells] to uninfected cells. Intercellular HIV transfer via TNT and/or TNT-like membrane conduits has been observed. These observations show that HIV-1 negative factor (Nef) protein is responsible for the formation of TNTs and/or TNT-like structures in infected cells to reach out to and infect non-CD4 cells. Nef is a 27- to 35-kDa HIV-1 accessory protein that alters the actin cytoskeletal organization and endocytic activity in T lymphocytes and dendritic cells. The proline-rich motif mediates the interaction with the SH3 domain of members of the Src family kinase family and Vav and the interaction induces actin cytoskeleton remodeling, endosome formation and signaling, stimuli or condition.

Microtubules as part of the intracellular trafficking system are essential transporters for delivering the virion to the plasma membrane for release to another cell. But even strong inhibitors of microtubule formation are poor anti-viral drugs. Because when using direct intercellular connection through TNTs many stages of virion release can be avoided the virion rather than requiring embellishment with escape and re-entry components needs only the basic RNA genome encased in nucleoprotein and polymerase subunits. The budding and scission are only involved in escape and attachment and fusion mechanisms are only required for finding and entering a naïve cell. The packaging of lipids and proteins necessary for packaging for these processes can be avoided. Risks inherent in possible immune exposure to the immune system in the circulation are also avoided by the direct cell-cell transfer. Other viruses of extreme concern, such as African swine fever, Ebola, Herpes Simplex, Marburg filoviruses and Poxvirus Vaccinia encode viral factors or alter cell activation to induce formation of filopodia or TNT like structures. HIV-1, one of the most studied viruses and therefore a model for general viral metabolisms and available functions has been shown to create, in infected cells, both short and long classes of TNTs as well as filopodia. Accordingly, the presence of filopodia can often be taken as evidence that the virus under appropriate conditions will have capabilities to employ TNTs for infecting near neighbor cells.

Different pathogens use TNTs or TNT-like structures as “freeways” to propagate between cells. Blocking these infectious paths is one means of preventing disease spread and progression. The CDC42/IRSp53/VASP network negatively regulates TNT formation and thus interferes with TNT-mediated intercellular pathogen and/or mitochondrial, etc. transfer. On the other hand, elevating Eps8, an actin regulatory protein that also inhibits the extension of filopodia in neurons, increases TNT formation. Eps8-mediated TNT induction requires Eps8 bundling but not its capping activity. Thus, despite their structural similarities and some shared construction tools, filopodia and TNTs form through distinct molecular mechanisms. A switch in the molecular composition in common actin regulatory complexes is another stage that is critical in driving the formation of either type of these membrane protrusions.

Protein diseases, such as Alzheimer's (AD), Huntington's, scrapie prion protein (PrPSc) and Parkinson's diseases also are promulgated by misapplication of the TNT pathway. Progressive accumulation of specific protein aggregates is a common hallmark of these major protein mediated neurodegenerative diseases. There is insufficient evidence to state that mad cow disease or bovine spongiform encephalopathy transits between cells through TNTs. This aspect of the disease has not received its due attention. So, while the pattern of disease spread is consistent with possible TNT participation and TNT involvement is highly likely, other transit pathways such as exosomal transmission may also participate.

Inhibiting or otherwise altering TNT activity that has been co-opted by for example, a virus that has learned to avoid the danger of immune system present in the extracellular circulatory system by infecting neighbor cells through TNT interconnections.

Two mechanisms for TNT formation are discussed in the scientific literature. One, commonly referred to as the “ccnism”, involves two cells contacting each other and fusing transiently with subsequently retention of a thin thread of membrane while they move apart. The initial breach of the two bilayers is the apparent energy limiting step. This method would not require a soluble intercellular messaging signal. A second mechanism, commonly referred to as the actin driven protrusion mechanism, involves an active process based on the extension of a filopodium-like protrusion from one cell to another, with membrane fusion of the tip upon physical contact. In both these models, the application of an actin depolymerizing drug will strongly reduce TNT formation.

The ability of endogenously and exogenously expressed M-Sec, a protein sharing homology with Sec, a component of the exocyst complex, to positively regulate TNT formation is not fully understood with regard to its effects on the membrane breaching energetics. Nevertheless, regardless of the exact underlying mechanisms tools are available for controlling TNT initiation, formation, extension and switching off.

M-Sec induction of TNT formation involves its interaction with the GTPase Ras-related A protein (RalA) and the exocyst complex. Furthermore, the transmembrane major histo-compatibility complex (MHC) class III protein leucocyte specific transcript 1 (LST1) interacts with M-Sec and mediates the recruitment of RalA to the plasma membrane, promoting its interaction with the exocyst complex. This multi-molecular complex can contribute to the remodeling of the actin cytoskeleton and to the delivery of membrane at the site of TNT formation. p53 also plays a crucial role in the formation of TNTs at least in astrocytes and with epidermal growth factor and the Akt/mammalian target of rapamycin (mTor)/phosphatidyl-inositol 3-kinase (P13K) pathway.

However, cells that do not express M-Sec, such as the mouse neuronal CAD cell line and neurons, can still participate in TNT transfers. And p53-independent TNT formation occurs at least in rat pheochromocytoma PC12 cells and in acute myeloid leukemia cells. Thus, a variety of tools exists for use when targeting cells to increase or to decrease their exchanged throughputs.

Another similarity that TNTs share with filopodia and their small diameter outcroppings is the requirement of actin for the protrusion. Expressing either of two filopodia inducers, the vasodilatator-stimulated phosphoprotein (VASP) and fascin, decreases the number of TNT-connected cells. But myosin X, another filopodial inducer, stimulates the formation of TNTs and intercellular transfer of vesicles. Thus, we have similar tools that can be interchanged for the desired effects.

RalA is another useful regulator for TNT formation. RalA binds filamin (a protein that cross links actin filaments) to promote TNT formation MHC class III protein, LST1, is also a key regulator of TNT formation. LST1 facilitates assembly of molecular machinery responsible for tunneling nanotube formation. LST1 induces nanotube formation though recruitment of RalA GTPase to the plasma membrane and there promoting its interaction with the exocyst complex. LST1 also recruits the actin-crosslinking protein filamin to the plasma membrane and thus interacts with M-Sec, myosin and myoferlin.

TNTs formation and stability is energetically possible only over a specific range of the cell membrane curvature. Lipid rafts are small (10-200 nm), heterogeneous, highly dynamic, cholesterol, and sphingolipid enriched domains that compartmentalize cellular processes by serving as organizing centers for the assembly of signaling molecules, influencing membrane fluidity and membrane protein trafficking, and regulating neurotransmission and receptor trafficking. Interactions between I-BAR proteins of lipid rafts and actin filaments are important factors for influencing the cell membrane curvature.

TNTs will grow from stressed cells toward non-insulted cells. S100A4 putative receptor, the receptor for advanced glycation end products (RAGE), is involved in TNT guidance but its mechanics of interactions are unclear.

NK cells can recognize and help eliminate microbial infection or tumor transformation. NK cell activity is regulated by a variety of both inhibitory and activating receptors with cognate ligands expressed on target cells. NKG2D can be used to recognize and target stress-induced ligands, e.g., MHC class I chain-related protein A (MICA), on the surface of a neighbor cell. And, DAP10 is a signaling actor that associates with the activating receptor NKG2D. DAP10, NKG2D, and MHC class I chain-related protein A (MICA) at the tip of nanotubes of human NK cells can induce immune responses in target cells.

NK cells are another cytotoxic lymphocyte that recognizes and contributes to the elimination of microbial infection or tumor transformation. Activation of NK cells is regulated by engagement of a variety of inhibitory and activating receptors with cognate ligands expressed on target cells. NKG2D as an activating receptor on NK cells, recognizes stress-induced ligands, such as MHC class I chain-related protein A (MICA), at the surface of other cells. Moreover, DAP10 is the signaling adaptor that associates with the activating receptor NKG2D. It showed that the accumulation of DAP10, NKG2D, and MHC class I chain-related protein A (MICA) at the tip of nanotubes of human NK cells can induce immune responses in target cells.

NK cells and in some cases macrophages build a specific form of TNT, one with microtubules. These are similar in polymerization effect and in some manners are reminiscent of intracellular protein bridges. In both microtubule based and F-actin based structures, Ca⁺⁺ binding is involved in the actomyosin within the TNT actively driving or transported cargo step by step, as the Ca⁺⁺ binds and unbinds the actin, through the TNT from one cell to the other. The process is directed and rapid i.e., significantly more rapid than diffusion. The requirement for Ca⁺⁺ in transport results in Ca⁺⁺ flux generally along its concentration gradient which can be controlled by IP3 release of Ca⁺⁺ from the ER or by alternative means of increasing Ca⁺⁺ concentration.

TNTs are useful for unidirectional and/or bidirectional intercellular transfer of cellular contents including proteins, Golgi vesicles, and mitochondria in human cancer. The presence of heteroplasmic mitochondrial DNA mutations implicates TNTs in the transfer of genetic change that may lead to tumor heterogeneity. MicroRNAs (miRNAs) can transfer between cells via TNTs. Thus, cultured cells removed from the body, engineered and returned might be useful for modifying expression in other cells though TNTs.

Several substances are available to choke off TNT events. For example, everolimus, a 40-O-(2-hydroxyethyl) derivative of sirolimus acts similarly to sirolimus as an inhibitor of mammalian target of rapamycin (mTOR) and IL-2 production. Metformin exhibits a strong and consistent anti-proliferative action on several cancer cell lines and shows a reliable antitumoral effect in animal models. Both metformin and everolimus can suppress TNT formation to and between cancer cells. These and similar compounds or derivatives may be used to thwart disease progression through TNTs.

The various methods can be used individually, in sequence or in combinations to accentuate TNT communications when desired. But when these TNTs are predominantly harming the host organism, methods that interfere with TNT formation or stability or limit size of the TNT pore to exclude passage of, for example, an intracellular parasite, can be applied.

1. A method of limiting spread of disease within a macroorganism, said method comprising inhibiting intercellular transport function of tunneling nanotubules (TNTs).

2. The method of claim 1 wherein the disease is spread by a virus.

3. The method of claim 2 wherein the virus is selected from the group consisting of: HIV-1, African swine fever, Ebola, Herpes Simplex, Influenza, Marburg filoviruses and Poxvirus Vaccinia.

4. The method of claim 1 wherein said inhibiting occurs between a time of suspected exposure to said disease and a time where the microorganism has developed protective antibody titer.

5. The method of claim 1 wherein the disease is a protein mediated disease.

6. The method of claim 5 wherein the disease is selected from the group consisting of: Alzheimer's (AD), Huntington's, scrapie prion protein (PrPSc) and Parkinson's diseases.

7. The method of claim 1 wherein said inhibiting comprises administering a compound selected from the group consisting of: pifithrin-α/(PFTα) HBr, reACp53, pifithrin-μ, NSC348884, everolimus and sirolimus.

8. A method of augmenting cyto-damaging or cytotoxic therapy, said method comprising inhibiting intercellular transport function of tunneling nanotubules (TNTs).

9. The method of claim 8 wherein said inhibiting comprises administering a compound selected from the group consisting of: pifithrin-α/(PFTα) HBr, reACp53, pifithrin-μ, NSC348884, everolimus and sirolimus.

10. The method of claim 9 wherein said inhibiting comprises administering to said macroorganism a preparation comprising exosomes.

11. The method of claim 10 wherein said administering to said macroorganism a preparation comprising exosomes involves: i) an isolation step to concentrate plasma membrane from lysed cells; ii) a vesiculation step wherein selected contents are incorporated in said preparation comprising exosomes; a packaging step wherein said preparation is prepared and formulated into a pharmaceutically acceptable composition; and iv) an incorporation step wherein said composition is incorporated into said macroorganism.

12. The method of claim 2 further comprising administering to said macroorganism a preparation comprising exosomes, said exosomes comprising at least one antiviral compound.

13. The method of claim 12 wherein said at least one antiviral compound comprises an inhibitory RNA directed against a requisite viral element.

14. The method of claim 12 wherein said at least one antiviral compound comprises an inhibitory RNA complementary to an RNA comprised in a virus selected from the group consisting of: HIV-1, African swine fever, Ebola, Herpes Simplex, Influenza, Marburg filoviruses and Poxvirus Vaccinia.

15. A method to improve metabolic function of an individual, said method comprising: modifying intercellular communication, in a macroorganism identified as a candidate for said modifying, between at least one stressed first cell and one or more surrounding second cell, said modifying involving modulation of presence or activity of at least one information transfer system selected from the group consisting of TNT and exosome.

16. The method of claim 15 wherein selection of said macroorganism identified as a candidate comprised data obtained from a bioassay.

17. The method of claim 16 wherein said bioassay is obtained from the group consisting of: a blood sample, a breath sample, a urine sample, a hair sample, a stool sample and a microbiome sample.

18. The method of claim 15 wherein selection of said macroorganism identified as a candidate comprised data obtained from at least one source selected from the group consisting of: a questionnaire, a survey, a blood sample, a breath sample, a urine sample, a hair sample, a stool sample, a microbiome sample, a group or population survey and an electronic sensor.

19. A method to improve health of a macroorganism, said method comprising: administering to said macroorganism a preparation of exosomes wherein at least a portion of which contains a metabolism improver selected from the group consisting of: a vitamin, a cofactor, a mitochondrial substrate, anandamide, an anandamide precursor, an inhibitor of PGD2 synthesis and an antioxidant.

20. The method of claim 19 wherein said metabolism improver selected from the group consisting of: Riboflavin (B2), L-Creatine, CoQ₁₀, L-arginine , L-carnitine, vitamin C, cyclosporin A, manganese, magnesium, carnosine, vitamin E, resveratrol, α-lipoic acid, folinic acid, fulvic acid, dichloroacetate, succinate, a prostaglandin (PG), a prostacyclin, a thromboxane, prostanoic acid, 2-arachidonoylglycerol, an NSAID, melatonin, cocaine, amphetamine, AZT, a mitophagic controlling compound, glutathione, β-carotene and a different carotenoid.

21. The method of claim 20 further comprising stimulating TNT initiation, elongation, growth, and or maintenance in said macroorganism.

22. The method of claim 19 wherein said preparation comprising exosomes comprises in at least a portion of said exosomes an inhibitory RNA targeting expression of a peptide selected from the group consisting of: f-actin, mTOR, RAGE, LST1, RalA GTPase, filamin, M-Sec, myosin, VASP, Akt, p53, CDC42, IRSp53 and myoferlin.

23. The method of claim 22 wherein said composition comprising exosomes comprises at least a portion of said exosomes containing at least one protein selected from the group consisting of: HSPA8, CD9, GAPDH, ACTB, CD63, CD81, ANXA2, ENO1, HSP9OAA1, EEF1A1, PKM2, AGO2, YWHAE, SDCBP, PDCD6IP, ALB, YWHAZ, EEF2, ACTG1, LDHA, HSP90AB1, ALDOA, MSN, ANXA5, PGK1 and CFL1.

24. The method of claim 15 modulation of presence or activity is monitored using data derived from a source selected from the group consisting of: a questionnaire, a survey, a blood sample, a breath sample, a urine sample, a hair sample, a stool sample, a microbiome sample, a group or population survey and an electronic sensor.
